(19) United States
(12) Patent Application Publication
(10) Pub. No.: US 2021/0022714 A1
(43) Pub. Date: Jan. 28, 2021

(54) NON-UNIFORM ROTATION DISTORTION (NURD) REDUCTION IN ULTRASOUND IMAGING DEVICES, SYSTEMS, AND METHODS

(71) Applicant: PHILIPS IMAGE GUIDED THERAPY CORPORATION, San Diego, CA (US)

(72) Inventors: Vladimir ZAGRODSKY, EL DORADO HILLS, CA (US); Bernhard STURM, DAVIS, CA (US); Nili KARMON, SACRAMENTO, CA (US); Kellen MOULTON, SAN MARCOS, CA (US); Arnoldo CURRLIN, MURRIETA, CA (US)

(21) Appl. No.: 16/898,562

(22) Filed: Jun. 11, 2020

Related U.S. Application Data
(60) Provisional application No. 62/878,978, filed on Jul. 26, 2019.

(51) Int. Cl. A61B 8/08; A61B 8/12; B06B 1/02

(52) U.S. Cl. CPC A61B 8/5207; B06B 1/0207; A61B 8/12

(57) ABSTRACT

Ultrasound image devices, systems, and methods are provided. An ultrasound imaging system, comprising an intraluminal imaging device configured to be positioned within a body lumen of a patient, the intraluminal imaging device comprising a rotatable, flexible elongate drive cable; and an ultrasound transducer disposed at a distal portion of the drive cable, the ultrasound transducer configured to transmit ultrasound energy into the body lumen and to receive ultrasound echoes associated with the body lumen; and a processor circuit in communication with the intraluminal imaging device and configured to receive, from the ultrasound transducer, signal data corresponding to the received ultrasound echoes; normalize the signal data; remove a low frequency signal component from the normalized signal data; determine a displacement for one or more scanlines of the signal data after removing the low frequency signal component; and generate an image of the body lumen based on the displacement.

### CROSS-REFERENCE TO PRIOR APPLICATIONS

This application claims the benefit of U.S. Provisional Patent Application No. 62/878,978, filed Jul. 26, 2019 which is hereby incorporated by reference herein.

### TECHNICAL FIELD

The present disclosure relates generally to intraluminal imaging devices, in particular, to providing image processing techniques to reduce the effects of non-uniform rotation distortion (NURD) in ultrasound images.

### BACKGROUND

Intravascular ultrasound (IVUS) imaging is widely used in interventional cardiology as a diagnostic tool for assessing a diseased vessel, such as an artery, within the human body to determine the need for treatment, to guide the intervention, and/or to assess its effectiveness. An IVUS device including one or more ultrasound transducers is passed into the vessel and guided to the area to be imaged. The transducers emit ultrasonic energy in order to create an image of the vessel of interest. Ultrasonic waves are partially reflected by discontinuities arising from tissue structures (such as the various layers of the vessel wall), red blood cells, and other features of interest. Echoes from the reflected waves are received by the transducer and passed along to an IVUS imaging system. The imaging system processes the received ultrasound echoes to produce a cross-sectional image of the vessel where the device is placed. IVUS imaging can provide detailed and accurate measurements of lumen and vessel sizes, plaque areas and volumes, and location of key anatomical landmarks. IVUS imaging allows physicians to evaluate the size of a lesion, select a treatment device (e.g., a stent) based on the evaluated lesion size, and subsequently evaluate the treatment success.

The two types of intravascular devices in common use today are solid-state and rotational. A conventional solid-state intravascular device may use an array of transducers (typically 64) distributed in close proximity around a circumference of a sheath, the sheath being an outer layer of the catheter. Also, an acoustic-matching path conducive to ultrasound wave propagation may be formed between the transducer and the sheath. The transducers are connected to an electronic multiplexer circuit. The multiplexer circuit selects transducers from the array for transmitting ultrasound signals and receiving reflected ultrasound signals. By stepping through a sequence of transmit-receive transducer pairs, the solid-state intravascular device can synthesize the effect of a mechanically scanned transducer element, but without moving parts. Since there is no rotating mechanical element, the transducer array can be placed in closer contact with blood and vessel tissue with minimal risk of vessel trauma, and the solid-state scanner can be wired directly to the IVUS imaging system with a simple electrical cable and a standard detachable electrical connector. In general, the need to flush the catheter with saline or other contrast media to form the acoustic-matching path is avoided.

On the other hand, a conventional rotational intravascular device may include a flexible drive cable that continually rotates inside the sheath of the catheter inserted into the vessel of interest. The drive cable may have a transducer disposed at a distal end thereof. The transducer is typically oriented such that the ultrasound signals propagate generally perpendicular to an axis of the catheter. In the typical rotational catheter, the sheath may be filled with fluid (e.g., saline) to protect the vessel tissue from the rotating drive cable and transducer while permitting ultrasound signals to freely propagate from the transducer into the tissue and back. As the drive cable rotates (e.g., at 30 revolutions per second), the transducer is periodically excited with a high voltage pulse to emit a short burst of ultrasound. The ultrasound signals are emitted from the transducer, through the fluid-filled sheath and sheath wall, in a direction generally perpendicular to an axis of rotation of the drive cable (i.e., the axis of the IVUS catheter). The transducer then listens for returning ultrasound signals reflected from various tissue structures, and the IVUS imaging system assembles a two-dimensional image of the vessel cross-section from a sequence of several hundred of these ultrasound pulse/echo acquisition sequences occurring during a single revolution of the drive cable and the transducer

However, ultrasound images obtained by the conventional rotational catheters may exhibit distortion due to non-uniform rotational distortion (NURD) experienced by the rotating drive cable. The distorted images are less effective at providing the required insight into the vessel condition. NURD may occur due to, for example, friction between the drive cable and the sheath that encloses the drive cable; friction between the sheath and the vessels through which the catheter travels through during use; non-symmetrical drive cable/transducer assembly that causes the drive cable to resist bending more at some angles than at other angles (when rotated, these asymmetries cause the drive cable to store more energy in some angular orientations and then to release that energy as the drive cable is rotated past that angle); the sheath and drive cable containing various bends and twists along its path to the vessel of interest; imperfect imbalance of the driving motor that provides the rotation of the drive cable, resulting in the transducer rotating at a non-uniform angular velocity even though one portion (e.g., the proximal portion) of the drive cable is rotated at a near-constant speed (because real actuators have limited torque, unlike ideal actuators). Further, catheters are often designed or configured to provide a greater flexibility for better deliverability through tortuous vessel path. However, catheters with a greater flexibility are typically more susceptible to NURD artifacts.

### SUMMARY

While existing IVUS imaging system have proved useful, there remains a need for improved systems and techniques for producing high quality ultrasound images with a minimal distortion (e.g., minimal NURD) for effective diagnostic assessment. Embodiments of the present disclosure provide techniques for NURD reduction in ultrasound images via image processing. For example, an intraluminal imaging device may include an ultrasound transducer positioned at a distal portion of a rotatable, flexible elongate drive cable. The drive cable may be coupled to a rotational source (e.g., a motor) that causes the ultrasound transducer to rotate during data acquisition. The disclosed embodiments perform speckle enhancement prior to detecting NURD-induced variations in the speckle pattern from the acquired ultrasound data (e.g., scanlines). The speckle enhancement may include applying intensity normalization and/or a background subtraction to an acquired ultrasound image data frame. The normalization can provide an about uniform average amplitude (e.g., intensity) for the speckle pattern. The background subtraction can suppress low frequency intensity variations in the image frame while maintaining the speckle pattern. As such, the speckle enhancement effectively emphasizes the speckle pattern against the background image. The detection of NURD-induced variations in the speckle pattern may include tracking or counting a number of consecutive neighboring samples (e.g., at a certain imaging depth) having intensities above the background intensity and/or a number of consecutive neighboring samples having intensities below the background intensity. The speckle detection and/or evaluation may include determining metrics based on the tracking. The metrics may indicate whether a rotational slowdown and/or a speedup occurred at the imaging device while the data is acquired. An ultrasound image can be constructed by interpolating and/or resampling the NURD affected scanlines based on the metrics. The interpolation and/or resampling can provide NURD reduction in the final image.

In one embodiment, an ultrasound imaging system includes an intraluminal imaging device configured to be positioned within a body lumen of a patient, the intraluminal imaging device comprising a rotatable, flexible elongate drive cable; and an ultrasound transducer disposed at a distal portion of the drive cable, the ultrasound transducer configured to transmit ultrasound energy into the body lumen and to receive ultrasound echoes associated with the body lumen; and a processor circuit in communication with the intraluminal imaging device and configured to receive, from the ultrasound transducer, first signal data corresponding to the received ultrasound echoes; normalize the first signal data by adjusting an intensity range of the first signal data; determine second signal data based on a removal of a low frequency signal component from the first normalized signal data; determine a displacement for one or more scanlines of the first signal data based on the second signal data; generate an image of the body lumen based on the displacement; and output the image to a display in communication with the processor circuit.

In some embodiments, wherein the displacement is associated with a rotational motion of the ultrasound transducer. In some embodiments, wherein the processor circuit configured to normalize the first signal data is further configured to apply a reference intensity map to the first signal data. In some embodiments, wherein the processor circuit configured to generate the image is further configured to perform brightness-mode (B-mode) processing on the first signal data based on a log compression map different from the reference intensity map. In some embodiments, wherein the processor circuit configured to determine the second signal data is further configured to apply a high-pass filter to the first normalized signal data. In some embodiments, wherein the processor circuit configured to determine the second signal data is further configured to determine an average intensity for at least a portion of the first normalized signal data; and subtract the average intensity from the first normalized signal data. In some embodiments, wherein the processor circuit is further configured to determine a metric for each scanline of the one or more scanlines based on the second signal data. In some embodiments, wherein a first scanline of the one or more scanlines includes a first sample at a first imaging depth and a second sample at a second imaging depth, and wherein the processor circuit configured to determine the metric is further configured to determine a first quantity of consecutive samples at the first imaging depth across neighboring scanlines of the first scanline comprising a first intensity value; determine a second quantity of consecutive samples at the second imaging depth across neighboring scanlines of the first scanline comprising the first intensity value; and determine the metric for the first scanline based on the first quantity and the second quantity. In some embodiments, wherein the processor circuit configured to determine the metric is further configured to apply at least one of an accumulation operation, an averaging operation, or a median operation to the first quantity and the second quantity. In some embodiments, wherein the first intensity value is associated with a background intensity threshold. In some embodiments, wherein the processor circuit is further configured to determine a rotational parameter of the ultrasound transducer associated with the one or more scanlines based on a corresponding metric. In some embodiments, wherein the processor circuit configured to determine the displacement is further configured to determine a lateral displacement for the one or more scanlines based on the rotational parameter. In some embodiments, wherein the processor circuit configured to generate the image is further configured to resample the one or more scanlines based on the displacement. In some embodiments, wherein the processor circuit configured to generate the image is further configured to perform brightness-mode (B-mode) processing on the one or more resampled scanlines.

In one embodiment, a method of ultrasound imaging includes receiving, at a processor circuit in communication with an intraluminal imaging device including an ultrasound transducer positioned at distal portion of a rotational, flexible elongate drive cable, first signal data corresponding to ultrasound echoes associated with a body lumen; normalizing the first signal data by adjusting an intensity range of the first signal data; determining second signal data based on a removal of a low frequency signal component from the first normalized signal data; determining a displacement for one or more scanlines of the first signal data based on the second signal data; generating an image of the body lumen based on the displacement; and outputting the image to a display in communication with the processor circuit.

In some embodiments, wherein the displacement is associated with a rotational motion of the ultrasound transducer. In some embodiments, wherein the normalizing the first signal data includes applying a reference intensity map to the first signal data, and the determining the second signal data includes applying at least one of a high-pass filter operation or an averaging operation to the first normalized signal data. In some embodiments, wherein a first scanline of the one or more scanlines includes a first sample at a first imaging depth and a second sample at a second imaging depth, and wherein the method further comprises determining a first quantity of consecutive samples at the first imaging depth across neighboring scanlines of the first scanline based on an intensity threshold; determining a second quantity of consecutive samples at the second imaging depth across neighboring scanlines of the first scanline based on the intensity threshold; and determining a metric for the first scanline based on the first quantity and the second quantity. In some embodiments, wherein the determining the metric includes applying at least one of an accumulation operation, an averaging operation, or a median operation to the first quantity and the second quantity. In some embodiments, the method further comprises determining a metric for each scanline of the one or more scanlines based on the second signal data; determining a rotational parameter of the ultrasound transducer associated with the one or more scanlines based on corresponding metrics, wherein the determining the displacement includes determining a lateral displacement for the one or more scanlines based on the rotational parameter; and wherein the generating the image includes resampling the one or more scanlines based on the lateral displacement.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a schematic diagram of an intraluminal ultrasound imaging system, according to aspects of the present disclosure.

FIG. 2 is a schematic diagram of a rotational intraluminal ultrasound imaging system, according to aspects of the present disclosure.

FIG. 3 is a schematic diagram of a rotational intraluminal imaging device, according to aspects of the present disclosure.

FIG. 4 is a schematic diagram illustrating a rotational ultrasound device operational scenario, according to aspects of the present disclosure.

FIG. 5 illustrates an ultrasound image including non-uniform rotation distortion (NURD) artifacts, according to aspects of the present disclosure.

FIG. 6 is a schematic diagram of an intraluminal ultrasound imaging system that implements NURD reduction, according to aspects of the present disclosure.

FIG. 7 is a schematic diagram of a NURD reduction component, according to aspects of the present disclosure.

FIG. 8 is a schematic diagram of a speckle enhancement scheme, according to aspects of the present disclosure.

FIG. 9 is a schematic diagram of a speckle enhancement scheme, according to aspects of the present disclosure.

FIG. 10 illustrates speckle normalization in ultrasound images, according to aspects of the present disclosure.

FIG. 11 illustrates binary thresholding in ultrasound images, according to aspects of the present disclosure.

FIG. 12 is a schematic diagram illustrating a speckle evaluation scheme, according to aspects of the present disclosure.

FIG. 13 is a schematic diagram illustrating a speckle evaluation scheme, according to aspects of the present disclosure.

FIG. 14 illustrates speckle metrics for a speckle enhanced image data frame, according to embodiments of the present disclosure.

FIG. 15 is a schematic diagram of a scanline resampling scheme, according to aspects of the present disclosure.

FIG. 16 illustrates NURD reduction in an ultrasound image, according to aspects of the present disclosure.

FIG. 17 is a schematic diagram of a processor circuit, according to embodiments of the present disclosure.

FIG. 18 is a flow diagram of an ultrasound imaging method with NURD reduction, according to aspects of the disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

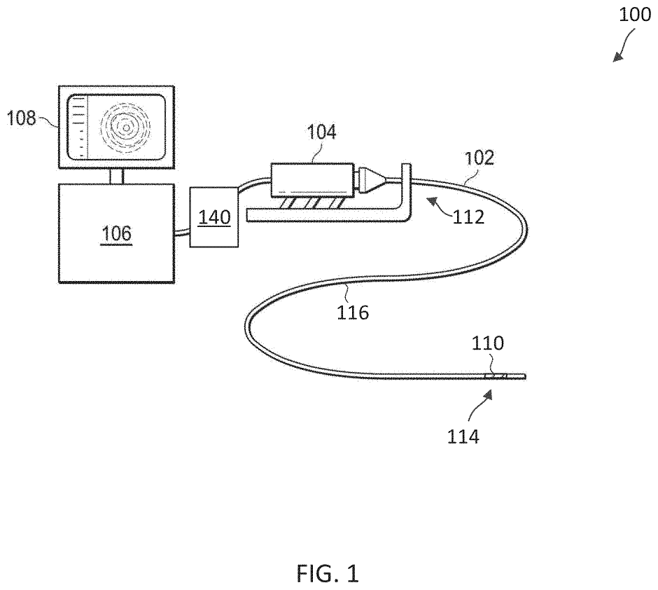

FIG. 1 illustrates an intraluminal ultrasound imaging system 100 according to aspects of the present disclosure. The system 100 may include an intraluminal imaging device 102, a patient interface module (PIM) 140, a processing system 106, and a display 108. Generally, the intraluminal imaging device 102 may be a catheter, a guide wire, or a guide catheter. The intraluminal imaging device 102 can be referred to as an interventional device and/or a diagnostic device. In some instances, the intraluminal imaging device 102 can be a therapeutic device. The processing system 106 may include various computing hardware and/or software components. In some instances, the processing system 106 may be a console, a computer, a laptop, a tablet, or a mobile device. The display 108 may be a monitor. In some embodiments, the display 108 may be an integrated component of the processing system 106.

The intraluminal imaging device 102 may include a flexible elongate member 116. sized and shaped for insertion into the vasculature of a patient. The flexible elongate member 116 may include a distal portion 114 and a proximal portion 112. The intraluminal imaging device 102 may include an imaging component 110 mounted at the distal portion 114 near a distal end of the intraluminal imaging device 102. The intraluminal imaging device 102 may be inserted into a body lumen or vessel of the patient. For example, the intraluminal imaging device 102 can be inserted into a patient's vessel to capture images of the structure of the vessel, measure the diameter and/or length of the vessel to guide stent selection, and/or measure blood flow in the vessel object.

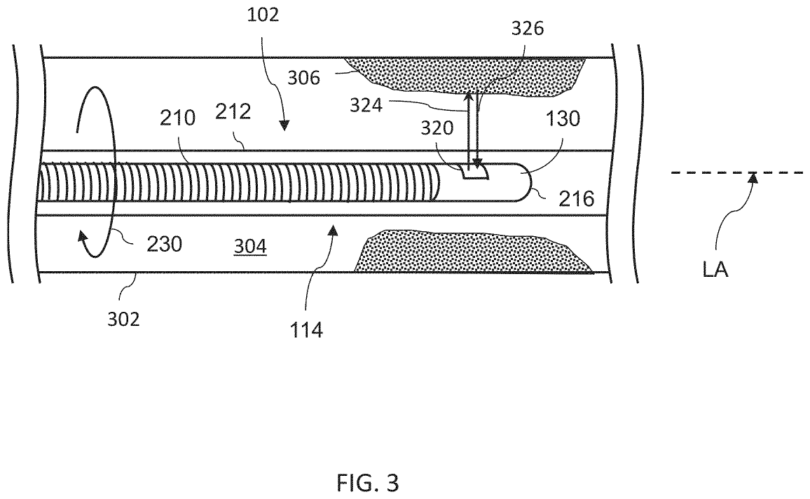

In an embodiment, the imaging component 110 may be or include a transducer (e.g., the transducer 320 in FIG. 3) or an acoustic element configured to emit ultrasonic energy towards a vessel object (e.g., a patient's vessel). In some embodiments, the imaging component 110 may include a piezoelectric micromachined ultrasound transducer (PMUT), capacitive micromachined ultrasonic transducer (CMUT), single crystal, lead zirconate titanate (PZT), PZT composite, other suitable transducer types, and/or combinations thereof. The emission of the ultrasonic energy may be in the form of pulses. The ultrasonic energy is reflected by tissue structures and/or blood flows in the vessel object surrounding the imaging component 110. The reflected ultrasound echo signals or echo responses are received by the ultrasound transducers in the imaging component 110. In some instances, the imaging component 110 may be configured for brightness-mode (B-mode) imaging to capture images of vessel structures or to measure vessel diameters and lengths for stent selection. In some other instances, the imaging component 110 may be configured for Doppler color flow imaging to provide blood flow measurements. In yet some other instances, the imaging component 110 may be configured to operate in a dual-mode to provide both B-mode imaging data and Doppler flow measurements.

For diagnosis and/or imaging, the transducer may include a center frequency between 10 MHz and 70 MHz, for example, including values such as 10 MHz, 20 MHz, 40 MHz, 45 MHz, 60 MHz, and/or other suitable values both larger and smaller. For example, lower frequencies (e.g., 10 MHz, 20 MHz) can advantageously penetrate further into an anatomy, such that more of the anatomy is visible in the ultrasound images. Higher frequencies (e.g., 45 MHz, 60 MHz) can be better suited to generate more detailed ultrasound images of a body lumen and/or fluid within the lumen. In some embodiments, the frequency of the transducer is tunable. For imaging, in some instances, the transducer can be tuned to receive wavelengths associated with the center frequency and/or one or more harmonics of the center frequency. In some instances, the frequency of the emitted ultrasonic energy can be modified by the voltage of the applied electrical signal and/or the application of a biasing voltage to the transducer.

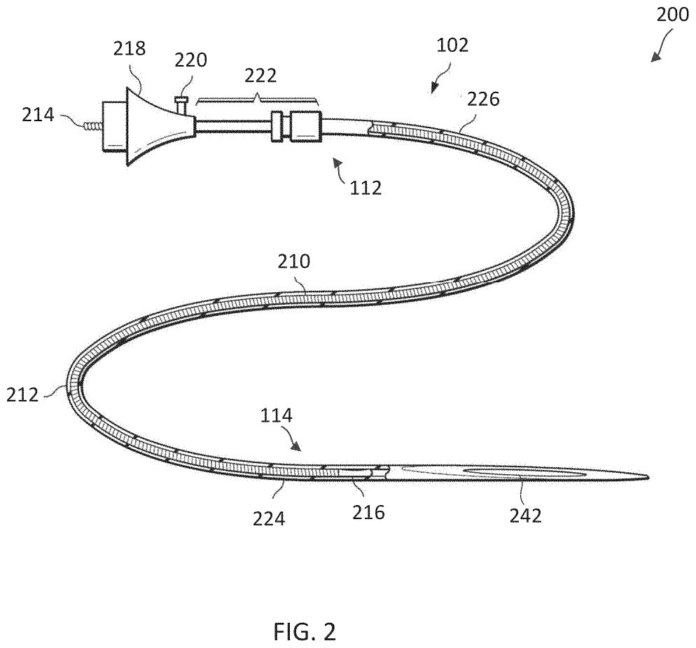

According to embodiments of the present disclosure, the intraluminal imaging device 102 is a rotational ultrasound device and the system 100 further comprises a movement device 104. The proximal portion 112 of the intraluminal imaging device 102 is mechanically and/or electrically coupled to the movement device 104. The movement device 104 includes one or more motors, associated circuitry, and/or other suitable components structurally arranged to impart rotational and/or longitudinal movement to one or more components of the intraluminal device 102, such as a drive cable (e.g., the drive cable 210 in FIG. 2). The movement device 104 can be referenced as a pullback device and/or a sled in some instances.

The movement device 104 is coupled to the PIM 140. The PIM 140 may allow delivery of DC supply voltages to the transducer circuitry at the imaging component 110. The PIM 140 may generate and/or provide the required sequence of transmit trigger signals and control waveforms to regulate the operation of the circuitry, and may process the amplified echo signals received over, for example, that same conductor pair. The PIM 140 may also supply the high- and low-voltage DC power supplies to support operation of the movement device 104 In that regard, the PIM 140 is structurally arranged to DC supply voltages to the circuitry of the intraluminal imaging device 102 across a rotational interface, using slip rings and/or the implementation of the active spinner technology described in U.S. Pat. No. 8,403,856, which is hereby incorporated by reference in its entirety. In some embodiments, the PIM 140 supplies AC voltage to the imaging component 110 using, e.g., a rotary transformer. While FIG. 1 illustrates the movement device 104 as a separate distinct component from the PIM 140, in some embodiments, the movement device 104 may be integrated with the PIM 140.

FIGS. 2 and 3 illustrate additional detail regarding the structure of a rotational ultrasound device. FIG. 2 is a schematic diagram of a rotational intraluminal imaging system 200, according to aspects of the present disclosure. The system 200 may correspond to a portion of the system 100. FIG. 3 is a schematic diagram of the rotational intraluminal imaging device 102, according to aspects of the present disclosure. FIG. 3 illustrates the rotational intraluminal imaging device 102 in situ in an anatomy 302. In some respects, the intraluminal imaging device 102 is similar to rotational IVUS catheters such as the Revolution® catheter available from Volcano Corporation and described in U.S. Pat. No. 8,104,479, or those disclosed in U.S. Pat. Nos. 5,243,988 and 5,546,948, each of which is hereby incorporated by reference in its entirety. In that regard, the rotational intraluminal imaging device 102 includes an imaging core 210 and an outer catheter/sheath assembly 212. The imaging core 210 includes a flexible drive cable or shaft that is terminated at the proximal end of the proximal portion 112 by a rotational interface 214 providing electrical and mechanical coupling to the PIM 140. The imaging core 210 can also include one, two, three, four, or more electrical conductors in communication with the transducer 320. The distal portion 114 of the flexible drive shaft of the imaging core 210 is mechanically coupled to a proximal portion of a transducer housing 216 containing the transducer 320, and associated circuitry.

The catheter/sheath assembly 212 includes a hub 218 that supports the rotational interface 214 and provides a bearing surface and a fluid seal between the rotating and non-rotating elements of the intraluminal imaging device 102. The hub 218 includes a luer lock flush port 220 through which saline is injected to flush out the air within the sheath 212 and fill the inner lumen of the sheath 212 with an ultrasound-compatible fluid at the time of use of the intraluminal imaging device 102. The saline or other similar flush is typically required since air does not readily conduct ultrasound. Saline also provides a biocompatible lubricant for the rotating drive cable of the imaging core 210. The hub 218 is coupled to a telescope 222 that includes nested tubular elements and a sliding fluid seal that permit the catheter/sheath assembly 212 to be lengthened or shortened to facilitate axial or longitudinal movement of the transducer housing 216 within an acoustically transparent window 224 of the distal portion of the intraluminal imaging device 102. In some embodiments, the window 224 is composed of thin-walled plastic tubing fabricated from material(s) that readily conduct ultrasound waves between the transducer and the vessel tissue with minimal attenuation, reflection, or refraction. A proximal shaft 226 of the catheter/sheath assembly 212 bridges the segment between the telescope 222 and the window 224, and is composed of a material or composite that provides a lubricious internal lumen and optimum stiffness, but without the need to conduct ultrasound. The guidewire entry/exit port 242 is provided at the distal portion of the intraluminal imaging device 102 in the illustrated embodiment.

The movement device 104 rotates the drive cable of the imaging core 210 (e.g., in a clockwise or counter-clockwise direction 230) inside the polymer/plastic sheath 212 inserted into lumen 304 of the anatomy 302. Rotation of the drive cable 210 causes corresponding rotation of the housing 216, which is mechanically coupled to the drive cable. The transducer 320 is fixedly secured to the housing 216, and correspondingly rotate with the drive cable 210. The transducer 320 is oriented such that the respective ultrasound beams 324, 326 propagate generally perpendicular to the longitudinal axis LA of the intraluminal imaging device 102. The fluid-filled sheath 212 protects the tissue of the anatomy from the spinning transducer 320 and the driveshaft 210 while permitting ultrasound signals (e.g., the ultrasound beams 324, 326) to freely propagate. As the driveshaft 210 rotates (e.g., at 30 revolutions per second), the transducer 320 is selectively and/or periodically excited with a high voltage pulse to emit a burst of ultrasound energy. For imaging, the transducer 320 listens for the returning ultrasound echoes 326 reflected from various tissue structures of the anatomy 302. As an example, the anatomy 302 may include an occlusion 306, where the returning ultrasound echoes 326 may include structural information associated with the occlusion 306. Based on the IVUS imaging data obtained by the transducer 320, the processing system 106 assembles a two-dimensional image of the vessel cross-section from a sequence of several hundred of these ultrasound pulse/echo acquisition sequences occurring during a single revolution of the transducer 320.

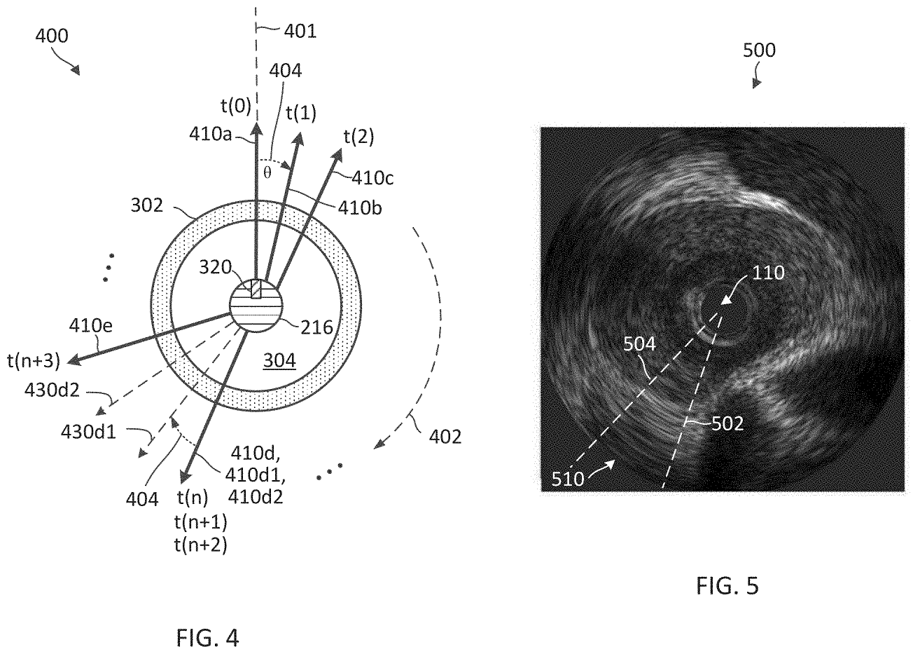

FIG. 4 illustrates a rotational ultrasound device operational scenario 400, according to aspects of the present disclosure. The scenario 400 may correspond to an operational scenario of the system 100 and/or 200. In the scenario 400, the intraluminal imaging device 102 is inserted into an anatomy 302 (e.g., a vessel object), where the imaging component 110 (e.g., the transducer 320) is positioned within the lumen 304 of the anatomy 302. FIG. 5 illustrates an ultrasound image 500 including NURD artifacts, according to aspects of the present disclosure. The image 500 may correspond to an image obtained from the scenario 400.

Referring to FIG. 4, the transducer 320 is disposed within the housing 216. The housing 216 includes an opening to allow propagation of ultrasound waves (e.g., the ultrasound beams 324, 326). The housing 216 is coupled to a drive cable (e.g., the driveshaft 210). Rotation of the drive cable causes corresponding rotation of the housing 216, and therefore the transducer 320. During data acquisition, the drive cable is driven by a movement device (e.g., the movement device 104) to cause the housing 216 and the transducer 320 to rotate, for example, in a clockwise direction 402. Alternatively, the drive cable can be configured to cause the housing 216 and the transducer 320 to rotate in a counter-clockwise direction. As the transducer 320 rotates, the transducer 320 is configured to emit an ultrasound signal and listen for an ultrasound echo reflected back from the tissue structure of the anatomy 302 and/or fluid flow within the lumen 304. The ultrasound echo may provide a scanline, which may be used to construct a cross-sectional image 500 of the anatomy 302.

In an example, the transducer 320 sweeps through a plurality of angular positions, for example, in steps of a rotational angle θ (shown as 404), in a clockwise direction. The angle 404 may be any suitable value depending on the embodiments. In some embodiments, the angle 404 may be about 360/512 degrees to provide about 512 scanlines per revolution. For example, the last 512^{th} position in a full revolution takes place at the time t(511) and provides a scanline at −θ degree from the axis 401. In general, the angle 404 may be about 360/N degree, where N is the number of scanlines per revolution. The N scanlines acquired by the transducer 320 may form an image frame of the anatomy 302 as shown in the image 500.

As an example, at time t(0), the transducer 320 is positioned at an angular position of about 0 degree with respect to the axis 401 to provide a scanline 410a. At time t(1), the transducer 320 is positioned at an angular position of about 0 degree with respect to the axis 401 to provide a scanline 410b at about θ degree. At time t(2), the transducer 320 is positioned at an angular position of about 2×θ degree with respect to the axis 401 to provide a scanline 410c at about 2×θ degree.

The rotational motion of the transducer 320 may continue at a constant speed to step through the rotational angle θ at each unit time. For example, at time t(n), the transducer 320 is positioned at an angular position of about n×θ degrees with respect to the axis 401 to provide a scanline 410d at about n×θ degrees.

As described above, a rotational ultrasound device may not rotate at a uniform speed throughout data acquisition, for example, due to an imperfect balance of the driving motor at the movement device 104 that provides the rotational motions and/or the intraluminal imaging device 102 having to traverse a tortuous catheter path through the vessel tree to reach the anatomy 302. Further, some intraluminal imaging catheters or devices may be designed to provide a great flexibility for delivery through the vessel tree. However, the greater the flexibility of the catheter, the more susceptible to NURD in the resultant ultrasound images. As such, the transducer 320 may step through a smaller rotational angle θ at one time and a larger rotational angle θ at another time.

As an example, after time t(n), the transducer 320 stops to rotate at the expected speed. In other words, the rotational speed of the transducer 320 falls to about 0 and the transducer 320 remains at about the same angular position of n×θ degrees with respect to the axis 401 for a duration, for example, from about time t(n) to about t(n+2). As such, the ultrasound echoes received by the transducer 320 at time t(n+1) and t(n+2) may each form a scanline 410d1 and 410d2, respectively, at the same angular position of about n×θ degrees as the scanline 410d or over a narrow sector of the anatomy 302.

After time t(n+2), the transducer 320 may resume the rotational motion, for example, speed up and then continue at the expected speed. As shown, at time t(n+3), the transducer 320 is positioned at an angular position of about (n+3)×θ degrees with respect to the axis 401 as expected and provides a scanline 410e at about (n+3)×θ degrees. Subsequently, the transducer 320 may return to a constant rotational speed providing a rotation of about 0 degrees at each unit time. When the processing system 106 receives the ultrasound echo signals collected by the transducer 320, the processing system 106 may process the ultrasound echo signals to construct a cross-sectional view of the anatomy 302 as shown in the image 500. The processing system 106 may generate the image 500 with the assumption that the transducer 320 at the transducer 320 rotates at a uniform rotational speed providing an angular rotation of about θ degrees at each unit time. In other words, the processing system 106 may perform scan conversion based on the scanline 410d1 being positioned at an angular position of about (n+1)×θ degrees with respect to the axis 401 corresponding to the scanline 430d1 and the scanline 410d2 being positioned at an angular position of about (n+2)×θ degrees with respect to the axis 401 corresponding to the scanline 430d2.

The assumption of the uniform rotational speed and the momentary irregular or non-uniform rotational speed of the transducer 320 (e.g., from the time t(n) to t(n+2)) can thus cause NURD in the final image. As can be observed, the image 500 includes a sector 510 between the dashed lines 502 and 504 showing angular smearing in the speckle pattern (e.g., a distortion in the azimuthal direction), which is the effect of NURD. Referring to the scenario 400 of FIG. 4, the dashed line 502 may correspond to an angular position of about nx0 degrees with respect to the axis 401 and the dashed line 504 may correspond to an angular position of about (n+3)×θ degrees with respect to the axis 401.

For simplicity of illustration and discussion, FIG. 4 illustrates a stoppage of the rotation between time t(n) to t(n+3), NURD effect can occur when the transducer 320 is rotated at a slower speed or a faster speed than the expected uniform rotational speed. An under-speed or slowdown of the rotation can cause lateral smearing as shown in the image 500. An over-speed or speed up of the rotation can cause lateral sharpening in the resultant ultrasound image. In addition, NURD may occur within multiple sectors within an ultrasound image. Further, while FIG. 4 illustrates about two scanlines being affected by the slowdown or stoppage of the rotations, the slowdown or stoppage of the rotations may affect a greater number of scanlines (e.g., about 3, 4, 5, 6 or more) or a less number of scanlines.

Several techniques including a cross-correlation-based technique as described in U.S. Pat. No. 8,956,299 and a frequency-averaging-based technique as described in U.S. Pat. No. 7,024025 may be applied to detect and reduce NURD in ultrasound images, each of which is hereby incorporated by reference in its entirety. However, the cross-correlation-based technique can be sensitive to image intensity variations caused by actual anatomical structures and/or artifacts characteristically present in an ultrasound image. The frequency-averaging-based technique can be sensitive to noise characteristically present in an ultrasound image. As such, the cross-correlation-based technique and the frequency-averaging-based technique may not always provide good NURD reduction performance. For example, the frequency-averaging-based technique may produce noise related distortion correction even when the imaging device is rotated at a uniform rotational speed.

Accordingly, the present disclosure provides signal processing techniques to detect NURD-induced variations in the speckle pattern (e.g., lateral smearing and/or lateral sharpening) in acquired scanlines and reduce the effect of NURD in the ultrasound image constructed from the scanlines. According to embodiments of the present disclosure, the signal processing operations may include speckle normalization and background subtraction to enhance the speckle pattern prior to detection of NURD-induced variations and NURD correction. The present disclosure may utilize various metrics to evaluate speckle patterns and determine lateral scanline displacements for NURD correction. The speckle pattern enhancement prior to the detection of NURD-induced variations and NURD correction can remove or reduce the dependencies from the image intensity and/or noise characteristics in the image for the speckle detection and NURD correction.

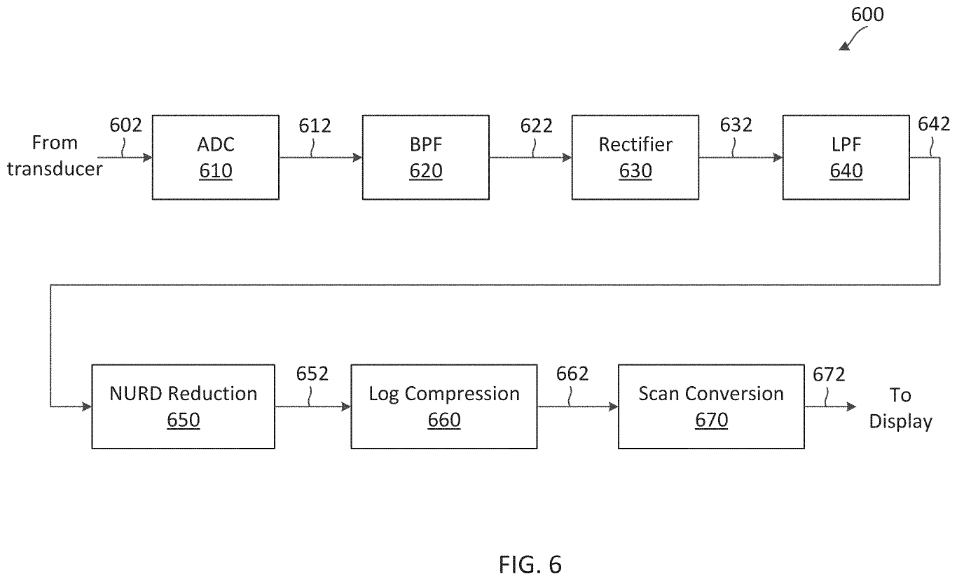

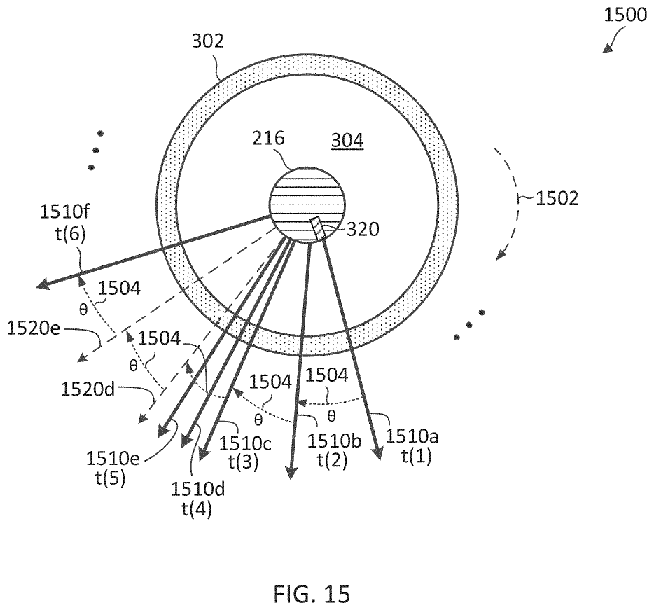

FIGS. 6-15 illustrate various NURD reduction mechanisms and corresponding results. FIG. 6 is a schematic diagram of an intraluminal ultrasound imaging system 600 that implements NURD reduction, according to aspects of the present disclosure. The system 600 may correspond to a portion of the system 100 and/or 200. The system 600 includes an analog-to-digital converter (ADC) 610, a bandpass filter (BPF) 620, a rectifier 630, a low-pass filter (LPF) 640, a NURD reduction component 650, a log compression component 660, and a scan conversion component 670. In some embodiments, the components of the system 600 may be implemented by the PIM 140. In some embodiments, the components of the system 600 may be implemented by the processing system 106. In some embodiments, the components of the system 600 may be distributed between the intraluminal imaging device 102, the PIM 140, and/or the processing system 106. The BPF 620, the rectifier 630, the LPF 640, the NURD reduction component 650, the log compression component 660, and the scan conversion component 670 can be implemented via a combination of hardware and software components. In some embodiments, the BPF 620, the rectifier 630, the LPF 640, the NURD reduction component 650, the log compression component 660, and/or the scan conversion component 670 can be implemented by a processor circuit as described in greater detail below in FIG. 15.

The ADC 610 is configured to receive raw analog ultrasound signal data 602 from a rotational ultrasound device (e.g., the imaging component 110 and/or the transducer 320). The analog ultrasound signal data 602 may include backscatter data received by the rotational ultrasound device at each angular position (e.g., at θ, 2×θ, 3×θ, . . . ) over one or more revolutions of the device. The ADC 610 is further configured to sample the analog ultrasound data 602 to provide digital ultrasound echo signal data 612. The digital ultrasound echo signal data 612 may include a plurality of scanlines (e.g., the scanlines 410) each including a sequence of real-valued RF samples along an imaging depth (e.g., a radial axis of the imaging device) as described in greater detail herein.

The BPF 620 is coupled to the ADC 610 and configured to apply a BPF to the digital ultrasound echo signal data 612 to produce signal data 622. The BPF may be configured with cutoff frequencies such that noise outside frequencies of interest is reduced in the output signal data 622.

The rectifier 630 is coupled to the BPF 620 and configured to convert the real-valued RF samples in the signal data 622 to baseband (BB) signal data 632 including complexed in-phase, quadrature-phase (IQ) pairs. The rectifier 630 may perform down-conversion, low-pass filtering, and/or decimation. The down-conversion converts the RF output signal data 622 from the RF to BB, for example, by down-mixing the RF signal data 602 with two sinusoidal signals with a 90 degrees phase difference. The LPF 640 is coupled to the rectifier 630 and configured to apply a LPF to the signal data 632 to remove side lobes or noise outside the desired frequency bandwidth.

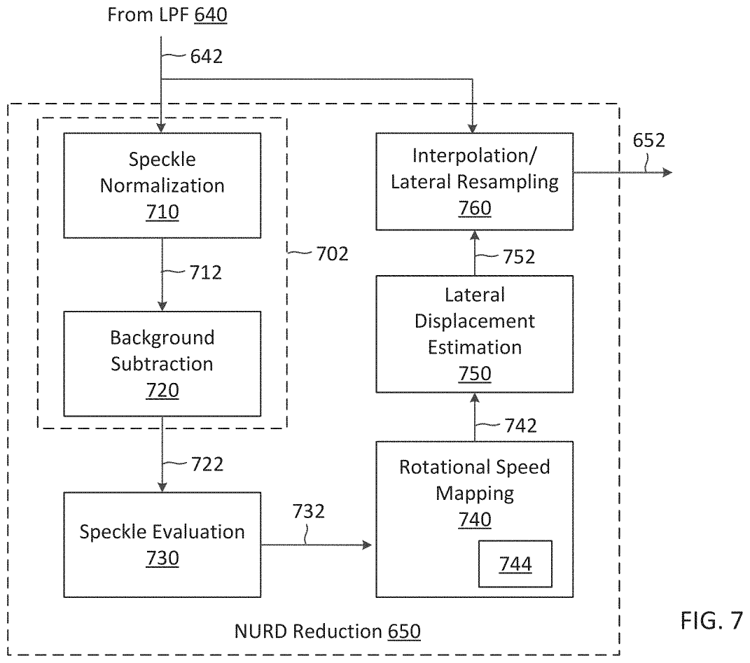

The NURD reduction component 650 is coupled to the LPF 640 and configured to perform NURD reduction on the signal data 642 and outputs NURD corrected or reduced signal data 652, as described in greater detail below in FIGS. 7-15.

FIG. 7 is a schematic diagram of the NURD reduction component 650, according to aspects of the present disclosure. The NURD reduction component 650 includes a signal pre-conditioning component 702, a speckle evaluation component 730, a rotational speed mapping component 740, a lateral displacement estimation component 750, and an interpolation/lateral resampling component 760.

The signal pre-conditioning component 702 is configured to enhance speckle pattern in the ultrasound echo signal data 642 output by the LPF 640. The signal pre-conditioning component 702 includes a speckle normalization component 710 coupled to a background subtraction component 720.

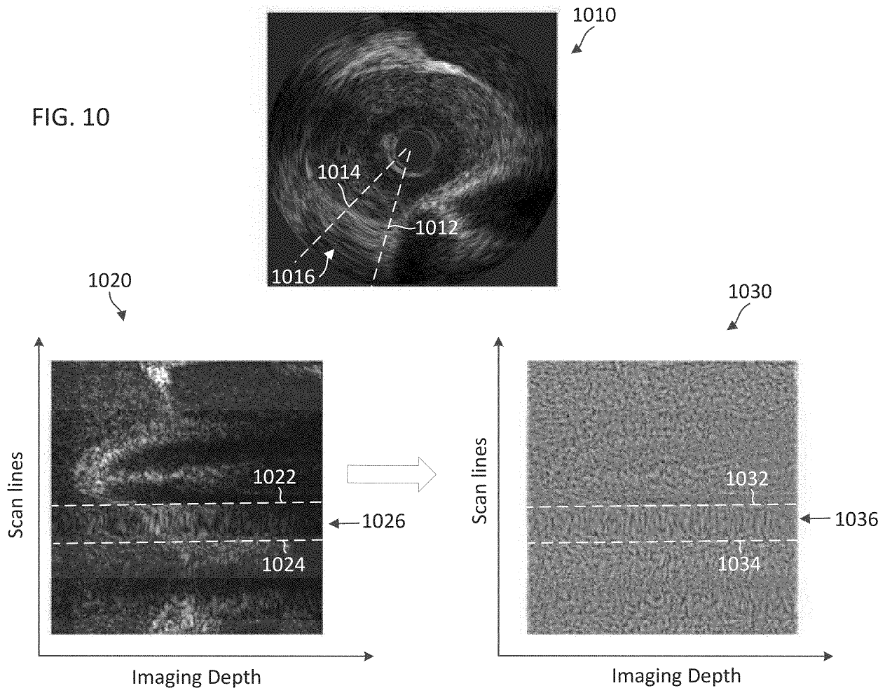

The speckle normalization component 710 is configured to shape the signal data 642 such that speckles in the signal data 642 may include a uniform average amplitude through the entire range of the underlying image of interest. In an embodiment, the speckle normalization component 710 implements a mapping function to provide an about uniform speckle average amplitude. In an embodiment, the mapping function is a non-linear intensity mapping function. In an embodiment, the mapping function is implemented as a table lookup with a logarithmic curve encoded into the lookup table. The speckle normalization component 710 outputs signal data 712, where portions of the signal data 712 corresponding to speckles may have an about uniform signal amplitude or intensity. In some examples, the input signal data 642 may include scanlines (e.g., the scanlines 410) with samples having 12-bit unsigned values and the output signal data 712 may include scanlines with samples having 8-bit unsigned values. The effect of speckle normalization is shown below in FIG. 10. The speckle normalization can reduce the dependencies of noise and/or signal characteristics in subsequent speckle evaluation (e.g., at the speckle evaluation component 730) as described in greater detailer herein.

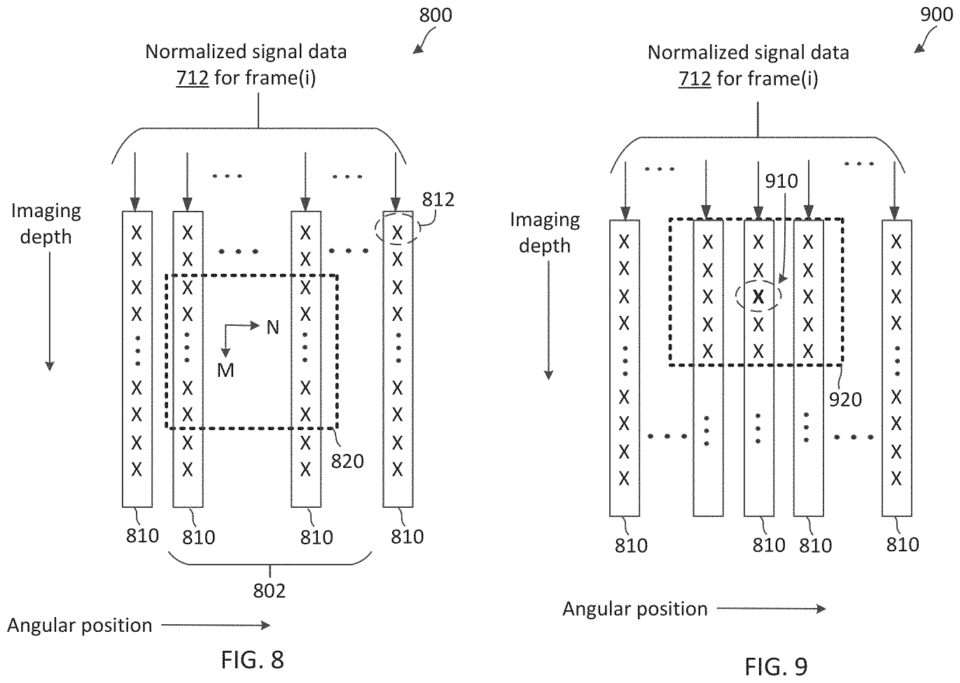

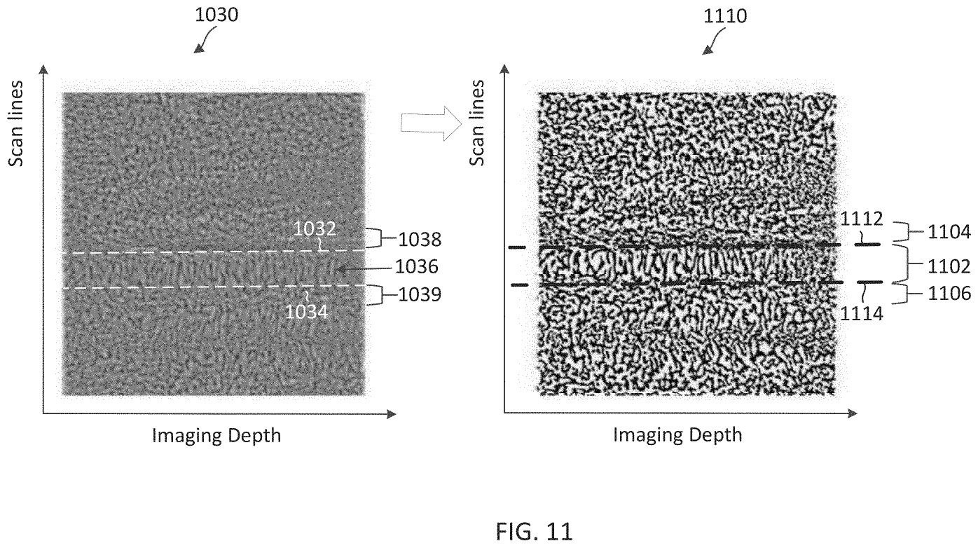

The background subtraction component 720 is configured to suppress low frequency intensity variations from the signal data 712 while maintaining the speckle pattern in place within the signal data 712. The background subtraction component 720 may be configured to include a two-dimensional high-pass filtering kernel as shown in FIG. 8 or a per-sample local image averaging kernel as shown in FIG. 9. The effect of speckle enhancement including the speckle normalization and background subtraction is shown below in FIG. 11. The background subtraction can enhance and/or emphasize the speckle pattern to assist subsequent speckle evaluation 730 and NURD reduction as described in greater detailer herein. In some examples, the background subtraction component 720 can produce output signal data 722 includes scanlines with samples that are binary values. For example, the output signal data 722 may have alternating groups of samples having intensity values of 1 (representing a black) and intensity values of 0 (represented white) as described in greater detail herein.

FIG. 8 is a schematic diagram of a speckle enhancement scheme 800, according to aspects of the present disclosure. The scheme 800 may be implemented by the background subtraction component 720. The scheme 800 performs speckle enhancement on a frame-by-frame basis. The scheme 800 receives a frame (e.g., represented by frame(i)) of speckle-normalized signal data 712. The frame of signal data 712 includes a plurality of scanlines 810. The scanlines 810 in the frame corresponds to scanlines received by a rotational ultrasound device (e.g., the rotational imaging component 110 and/or the transducer 320) as the rotational ultrasound device rotates in a full revolution (e.g., from 0, θ, 2×θ, 3×θ . . . to about (360−θ) degrees). Each scanline 810 is received when the transducer is at a particular angular position (e.g., at about 0 degree, 0 degrees, 2×θ degrees, . . . (360−θ) degrees). For example, the leftmost scanline 810 may be received first in time for the frame(i) when the transducer is positioned at an angular position of 0 degree and the rightmost scanline 810 may be received last in time for the frame(i) when the transducer is positioned at an angular position of about (360−θ) degrees. Each scanline 810 includes a sequence of samples 812 (shown as cross symbols) along an imaging depth or the radial direction.

The scheme 800 applies a two-dimensional convolutional kernel 820 to the samples 812. The convolutional kernel 820 may be high-pass-filter (HPF) kernel that suppresses low frequency intensity variations in the speckle-normalized signal data 712. The convolutional kernel 820 may have a size of N×M that operates over a group of samples 812 across N scanlines 810 and M samples 812 along the imaging depth, where M and N are positive integers. The convolutional kernel 820 may slide across the scanlines 810 and across the imaging depth for the filtering to produce speckle-enhanced signal data (e.g., the speckle-enhanced signal data 722). The dimensions of the convolutional kernel 820 may vary depending on the embodiments. In some examples, the device may receive about 512 scanlines 810 in a single revolution and each scanline 810 may include about 1000 samples 812 along the imaging depth. In such examples, the convolutional kernel 820 may have a dimension of about 16 scanlines×16 samples (e.g., N and M may be about 16).

In some examples, the scheme 800 may perform the filtering operations by considering scanlines 810 that are at the edges or margins of the frame. For example, when the scheme 800 filters a first scanline 810 received for a current frame (e.g., frame(i)), the scheme 800 may apply padding corresponding to lateral marginal scanlines 810 from a previous frame (e.g., frame(i−1)). Since the rotational motion of the rotational ultrasound device is continuous, a first scanline 810 of the current frame is immediately after a last scanline 810 of the previous frame. As such, the convolution operation upon such padding can be performed over a continuous sequence of scanlines 810. In some examples, the scheme 800 may not include the marginal scanlines 810 in the filtering as there may not be need for processing the entire radial range in the following steps. The highly saturated near-field zone and the mostly noisy far outskirts can be cropped out in order to keep processing of the most informative radial portion 802 of the scanlines 810.

FIG. 9 is a schematic diagram of a speckle enhancement scheme 900, according to aspects of the present disclosure. The scheme 900 may be implemented by the background subtraction component 720. The scheme 900 is described using the same speckle-normalized signal frame structure as in the scheme 800, and may use the same reference numerals as in FIG. 8 for simplicity sake. The scheme 900 utilizes an averaging kernel instead of a high-pass filtering kernel as in the scheme 800. The scheme 900 applies a two-dimensional averaging kernel 920 to the speckle-normalized signal data 712 on a frame-by-frame basis. The averaging kernel 920 may span a number of scanlines 810 laterally and a number of samples 812 along the imaging depth. For simplicity of illustration and discussion, FIG. 9 illustrates the averaging kernel 920 having dimensions of 3 scanlines×3 samples. However, the averaging kernel 920 may have any suitable dimensions, for example, spanning a greater number of scanlines 810 or less number scanlines 810 and/or spanning a greater number of samples 812 or less samples 812. The averaging kernel 920 computes an average signal amplitude value for samples 812 within the kernel 920. After applying the averaging kernel 920, the scheme 900 subtracts the average signal amplitude value from the center sample (shown as 910) within the kernel 920 to provide speckle-enhanced signal data (e.g., the speckle-enhanced signal data 722).

In general, the convolutional kernel 820 and the averaging kernel 920 may have dimensions exceeding a typical speckle size and be able to cover several speckles in the lateral direction (e.g., across the scanlines 810) and/or in the radial direction (e.g., along the imaging depth).

FIG. 10 illustrates speckle normalization in ultrasound images, according to aspects of the present disclosure. FIG. 10 shows ultrasound images 1010, 1020, and 1030. The images 1010, 1020, and 1030 are acquired using a rotational ultrasound device (e.g., the rotational imaging component 110 and/or the transducer 320) in an imaging system (e.g., the systems 100, 200, and/or 600). The image 1010 shows a scan-converted cross-sectional view of an anatomy (e.g., the anatomy 302) without NURD reduction. The image 1010 may correspond to the image 500 in FIG. 5. The image 1010 includes a sector 1016 (between the dashed lines 1012 and 1014) with a speckle smearing pattern. The angular smearing sector 1016 may be similar to the sector 510 in the image 500. The smearing speckles are the result of NURD caused by an unstable rotational speed of the ultrasound device.

The image 1020 is an image of the anatomy before speckle normalization in a polar form. The image 1020 may include scanlines corresponding to scanlines in the image 500 before B-mode processing (e.g., log compression) and/or scan conversion are applied to generate the image 500. The image 1030 is an image of the anatomy after speckle enhancement (e.g. normalization and background subtraction) in a polar form. The x-axes represent samples (e.g., the samples 812) along the imaging depth or radial direction, and the y-axes represent scanlines (e.g., the scanlines 810) across a frame (e.g., corresponding to angular positions from about 0 degree, 0 degrees, 2×θ degrees, . . . , to about 360 degrees).

The image 1020 may represent a frame of signal data (e.g., the signal data 642) before speckle normalization. The dashed lines 1022 and 1024 in the image 1020 correspond to the angular positions represented by the dashed lines 1012 and 1014, respectively, in the image 1010.

The image 1030 may represent a frame of signal data (e.g., the signal data 722) after applying speckle enhancement 702 to the image 1020, for example, using the speckle normalization component 710 of FIG. 7 and background subtraction 720 of FIG.8 or FIG.9. The dashed lines 1032 and 1034 in the image 1030 correspond to the angular positions represented by the dashed lines 1022 and 1024, respectively, in the image 1020.

The portion 1026 of the image 1020 between the dashed lines 1022 and 1024 and the portion 1036 of the image 1030 between the dashed lines 1032 and 1034 show a lateral smearing of the speckle pattern (the vertical lines) corresponding to the sector 1016 of the image 1010. The smearing of speckle pattern is caused by the slow down or stoppage of the rotational motion of the ultrasound device.

Comparing the images 1020 and 1030, the speckle-enhanced image 1030 includes a speckle pattern of a nearly uniform amplitude over the image 1030 with residual image features of bigger than speckle size and/or spatial scale being suppressed. As can be observed, the vessel walls corresponding to the large-sized image feature are clearly visible in the image 1020, but are hardly visible in the image 1030. The large-sized image features, such as the vessel walls and/or shadows, can be referred to as a background when viewing an image at a speckle scale. Thus, the background subtraction refers to the removal/suppression of large-sized features, such as walls and shadows seen in the image 1020. In other words, the speckles are emphasized and the anatomy is dimmed down in the image 1030.

FIG. 11 illustrates binary thresholding in ultrasound images, according to aspects of the present disclosure. The binary thresholding can facilitate speckle evaluation for the images. FIG. 11 shows ultrasound images 1030 and 1110. The image 1110 is captured using an imaging system similar to the systems 100, 200, and/or 600. Similar to the image 1030, the image 1110 is shown in a polar form, where the x-axis represents samples along an imaging depth and the y-axis represents scanlines. The image 1110 may represent a frame of signal data after applying a binary threshold to the image 1030. The portion 1102 of the image 1110 between the dashed lines 1112 and 1114 correspond to the portion of the image 1030 between the dashed lines 1032 and 1034.

The portion 1102 shows smearing of the speckle pattern (the vertical lines) caused by the rotational motion of the rotational ultrasound device being slowed down or stopped. The portion 1104 and 1106 of the image 1110 adjacent to the portion 1102 shows sharpening of the speckle pattern (the closely spaced lines) caused by the rotational motion of the rotational ultrasound device being sped up before and after the stoppage or slowdown of the rotational motion.

Comparing the image 1110 to the image 1030, the speckle pattern in the image 1110 is emphasized. The vertical lines (smearing of speckles) in the portion 1102 are visually distinct. The closely spaced lines (sharpening of speckles) in the portions 1104 and 1106 are also visually distinct, whereas corresponding portions 1038 and 1039 in the image 1030 exhibit less distinctive smearing and/or sharpening of the speckles. As illustrated in the image 1110, the speckle pattern includes alternating groups of samples with intensities above the background and below the background.

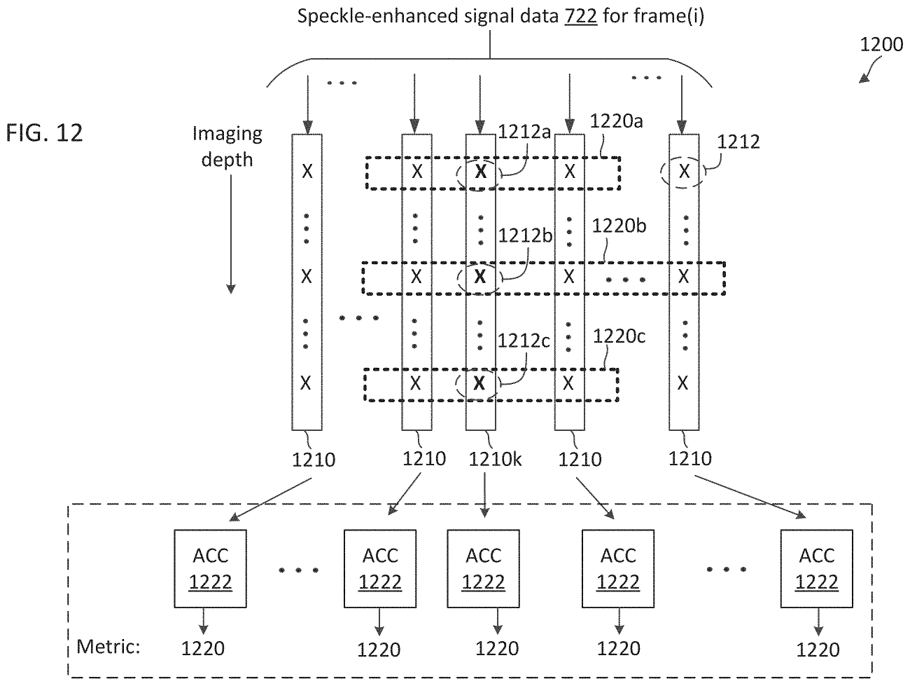

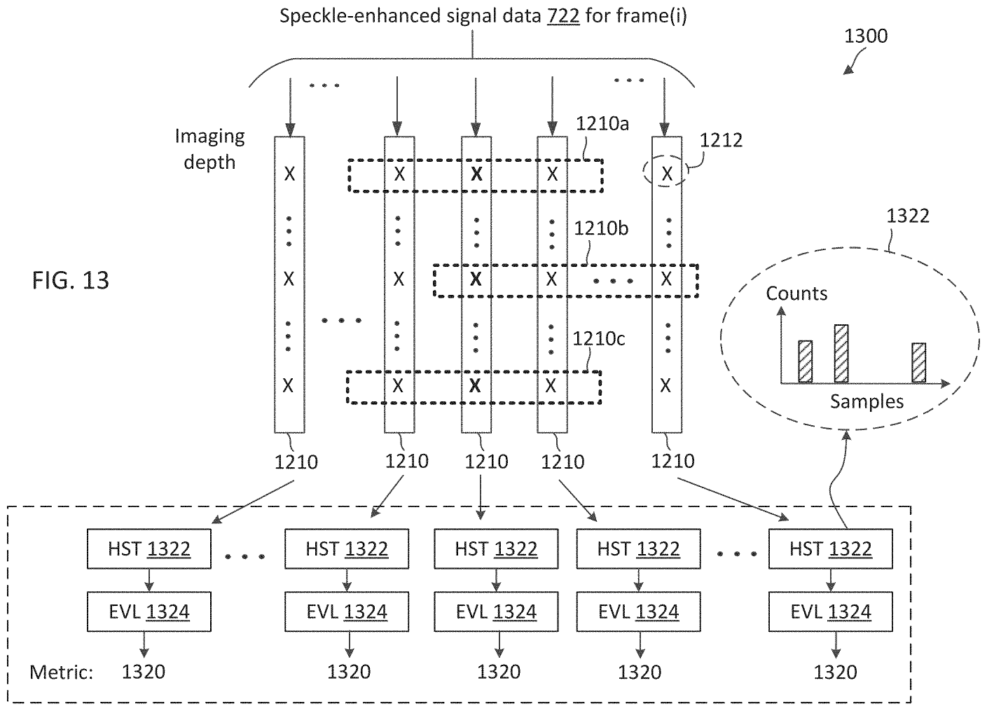

Returning to FIG. 7, the speckle evaluation component 730 is coupled to the signal pre-conditioning component 702 and configured to perform lateral evaluation of the normalized, enhanced speckle pattern in the signal data 722. The speckle evaluation component 730 is configured to perform lateral tracking of samples (e.g., the samples 812) across neighboring scanlines (e.g., the scanlines 810) based on the speckle pattern having alternating groups of samples with intensities above the background and below the background. The tracking may include binary thresholding as illustrated above in FIG. 11, and counting the number of samples being uninterruptedly above the background and/or being uninterruptedly below the background, as described in greater detail below in FIGS. 12 and 13. The speckle evaluation component 730 may compute speckle metrics 732 based on the tracking or counting to determine whether the rotational speed of the rotational ultrasound device was slowed down or sped up during data acquisition (while the frame of signal data 722 is acquired). The speckle evaluation component 730 may determine the speckle metric 732 using an accumulation-based scheme as shown in FIG. 12 or a histogram-based scheme as shown in FIG. 13.

FIG. 12 is a schematic diagram illustrating a speckle evaluation scheme 1200, according to aspects of the present disclosure. The scheme 1200 may be implemented by the speckle evaluation component 730. The scheme 1200 performs speckle evaluation on a frame-by-frame basis. The scheme 1200 receives a frame (e.g., represented by frame(i)) of the speckle-enhanced signal data 722. The frame of signal data 722 includes a plurality of scanlines 1210 across angular positions of a rotational ultrasound device (e.g., the rotational imaging component 110 and/or the transducer 320). Each scanline 1210 includes a sequence of samples 1212 along an imaging depth. The scanlines 1210 and the samples 1212 may correspond to the scanlines 810 and the samples 812, respectively, in FIGS. 8 and 9 after being processed by the background subtraction component 720.

The scheme 1200 performs direct counting of samples laterally (across angular positions) for each sample 1212 along each scanline 1210. The direct counting includes counting the number of consecutive neighboring samples 1212 across neighboring scanlines 1210 having intensities above the background intensity. Alternatively and additionally, the direct counting can include counting the number of consecutive neighboring samples 1212 across neighboring scanlines 1210 having intensities below the background intensity.

As an example, for the sample 1212a along the scanline 1210k, the scheme 1200 determines that neighboring samples within the window 1220a having intensities above the background intensity and/or below the background intensity and determine an integer count for the number of samples 1212 in the window 1220a. The evaluation of samples 1212 with respect to the background intensity (e.g., determining whether a sample value is above or below a threshold) may be similar to the binary thresholding described above with respect to FIG. 11. Similarly, for the sample 1212b along the scanline 1210k, the scheme 1200 determines that neighboring samples within the window 1220b having intensities above the background intensity and/or below the background intensity and determine an integer count for the number of samples 1212 in the window 1220b. For the sample 1212c along the scanline 1210k, the scheme 1200 determines that neighboring samples within the window 1220c having intensities above the background intensity and/or below the background intensity and determine an integer count for the number of samples 1212 in the window 1220c. The sizes of the windows 1220b may vary for each sample 1212 depending on the speckle pattern.

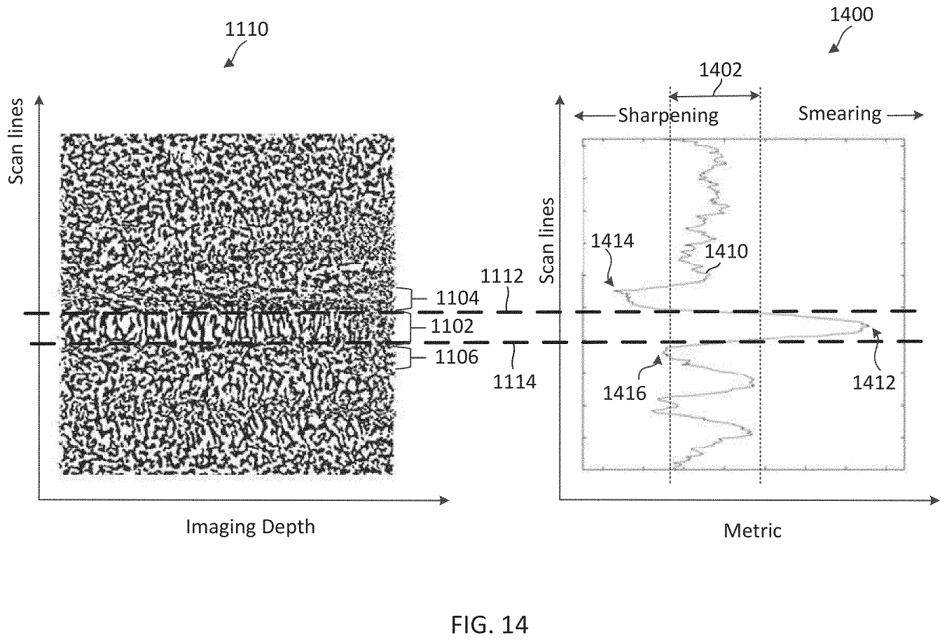

After the lateral tracking, the scheme 1200 generates a per-scanline metric 1220 (corresponding to the metrics 732) by applying an accumulator (ACC) 1222 to accumulate the per-sample integer counts for the samples 1212 along each scanline 1210. The scheme 1200 may determine whether the rotational ultrasound device was slowed down or sped up based on the metrics 1220 as described in greater detail below in FIG. 14.

FIG. 13 is a schematic diagram illustrating a speckle enhancement scheme 1300, according to aspects of the present disclosure. The scheme 1300 may be implemented by the speckle evaluation component 730. The scheme 1300 is described using the same speckled-enhanced signal frame structure and lateral speckle tracking or direct counting mechanisms as in the scheme 1200, and may use the same reference numerals as in FIG. 12 for simplicity sake. However, the scheme 1300 determines speckle metrics based on histograms of counters generated by the direct counting.

As described above, the direct counting outputs an integer count of speckles in a lateral direction (e.g., within the windows 1210a, 1210b, and 1210c) for each sample 1212 along each scanline 1210. The scheme 1300 generates a histogram (HST) 1322 of the integer counts for each scanline 1210. Each histogram 1322 may include a distribution of the integer counts across samples 1212 along a scanline 1210 as shown in the expanded view. The scheme 1300 determines a per-scanline metric 1320 (corresponding to the metrics 732) by applying an evaluator (EVL) 1324 to each histogram 1322. In an example, the evaluator 1324 computes an average value or a mean value for each histogram 1322 to produce a speckle metric 1320 for a corresponding scanline 1210. In an example, the evaluator 1324 computes a median value for each histogram 1322 to produce a speckle metric 1320 for a corresponding scanline 1210.

FIG. 14 illustrates speckle metrics for a speckle enhanced image data frame, according to embodiments of the present disclosure. FIG. 14 shows the image 1110 generated from a frame of speckle-enhanced signal data 722 and a speckle metric plot 1400 for the image 1110. FIG. 14 may use the same reference numerals as in FIG. 11 for simplicity sake. In the plot 1400, the x-axis represents metrics in some constant units, and the y-axis represents scanline corresponding to the scanlines of the image 1110. The plot 1400 shows speckle metrics 1410 for the scanlines (e.g., the scanlines 410, 810, and/or 1210) of the image 1110. The speckle metrics 1410 (e.g., the metrics 732, 1220, and/or 1320) may be generated by the speckle evaluation component 730 of FIG. 7.

The scanlines with metrics 1410 greater than a regular metric range 1402 (e.g., the metrics 1410 on the right side of the plot 1400) corresponds to scanlines that are affected by speckle smearing as shown by the arrow 1412. The regular metric range 1402 refers to the metric 1410 when the rotational ultrasound device is rotating at a uniform speed. The regular metric range 1402 may be determined based on calibration of the rotational ultrasound device or the type of the rotational ultrasound device. In some examples, the regular metric may span a range of values, rather than just one value as shown in FIG. 14. The scanlines with metrics 1410 below the regular metric range 1402 (e.g., the metrics 1410 on the left side of the plot 1400) corresponds to scanlines that are affected by speckle sharpening as shown by the arrows 1414 and 1416. As can be observed, the portion 1102 of the image 1110 between the dashed lines 1112 and 1114 affected by speckle smearing corresponds to the metrics 1410 indicating speckle smearing marked by the arrow 1412. The portions 1104 and 1106 of the image 1110 affected by speckle sharpening corresponds to the metrics 1410 indicating speckle sharpening marked by the arrows 1414 and 1416, respectively.

Returning to FIG. 7, the rotational speed mapping component 740 is coupled to the speckle evaluation component 730 and configured to determine the rotational speeds 742 of the rotational ultrasound device during data acquisition based on the speckle metrics 732 (e.g., the metrics 1220, and/or 1320). The rotational speed mapping component 740 may obtain a regular metric (e.g., within the regular metric range 1402) for the rotational ultrasound device. The rotational speed mapping component 740 may determine that the rotational ultrasound device experienced a slowdown of the rotational speed when the metrics 732 is above the regular metric (corresponding to a smearing of speckle pattern). The rotational speed mapping component 740 may determine that the rotational ultrasound device experienced a speedup of the rotational speed when the metrics 732 is below the regular metric (corresponding to a sharpening of speckle pattern).

In an embodiment, a particular mapping of the evaluated smearing or sharpening of the speckle pattern to the rotational speed of the rotational ultrasound device can be established by imaging calibrated phantoms in a particular image acquisition mode. The rotational speed mapping component 740 may map the rotational speed of the device based on the mapping. The stronger smearing of the speckle pattern is evaluated at a scanline, the slower is the momentary rotation when the scanline is acquired. Conversely, the stronger sharpening of the speckle pattern is evaluated at a scanline, the more accelerated is the momentary rotation when scanline is acquired.

In an example, the mapping may be stored in lookup table 744. The rotational speed mapping component 740 may perform a table lookup based on the speckle metrics 732. In some examples, different mapping tables 744 may be used for different types of rotational ultrasound devices and/or different image acquisition modes.

The lateral displacement estimation component 750 is coupled to the rotational speed mapping component 740 and configured to estimate angular spacing 752 between scanlines (e.g., the scanlines 410, 810, and/or 1210) in an image frame acquired by the rotational ultrasound device. As described above, the angular spacing between the scanlines may not be uniform due to the unstable rotational speed of the rotation ultrasound device during data acquisition. The lateral displacement estimation component 750 may determine the angular spacing 752 between among the scanlines based on the corresponding rotational speed 742 of the rotational ultrasound device when the scanlines are acquired. A slowdown in the rotational speed may correspond to a smaller angular spacing 752 whereas an acceleration in the rotational speed may correspond to a larger angular spacing 752.

The interpolation and lateral resampling component 760 is coupled to the lateral displacement estimation component 750. The interpolation and lateral resampling component 760 is configured to determine an interpolation factor based on the angular spacing 752. The interpolation factor may be a downsampling factor when the angular spacing 752 for scanlines (e.g., the scanlines 410, 810, and/or 1210) is small (affected by speckle smearing). Alternatively, the interpolation factor may be an upsampling factor when the angular spacing 752 is large when the angular spacing 752 for scanlines is large (affected by speckle sharpening).

The interpolation and lateral resampling component 760 may perform interpolation on scanlines in the signal data 642 based on the determined interpolation factor. In an example, the lateral displacement estimation component 750 may assign an angular spacing for a group of neighboring scanlines in a frame of signal data 642 and the interpolation and lateral resampling component 760 may perform the interpolation on the group of scanlines. The interpolation may be a piecewise-linear interpolation or any suitable interpolation techniques. The interpolation and lateral resampling component 760 may also apply smoothing to the group of scanlines to avoid inter-frame jitters and/or a seamline artifact at locations where a smearing sector is reduced.

The interpolation and lateral resampling component 760 may remap the interpolated scanlines to a uniform angular grid to produced uniformly spaced scanlines (e.g., the signal data 652). In some examples, the interpolation and lateral resampling component 760 may resample the non-uniformly spaced, interpolated scanlines into uniformly spaced scanlines that may be operated by a scan converter as described in greater detail herein. In some examples, the interpolation and lateral resampling component 760 may implement the interpolation with dynamic deformation of the scan conversion grid (instead of a regular uniform scan conversion grid) such that the scanlines (e.g., including polar samples) are mapped directly into the Cartesian space after the lateral displacement estimation. The interpolation, remapping, and/or resampling mechanisms are described in greater detail below in FIG. 15.

FIG. 15 is a schematic diagram of a scanline resampling scheme 1500, according to aspects of the present disclosure. The scheme 1500 is implemented by the interpolation and lateral resampling component 760. Similar to the scenario 400, the intraluminal imaging device 102 is inserted into an anatomy 302. During data acquisition, the transducer 320 disposed within the housing 216 is configured to rotate at a certain rotational speed, where the ultrasound transducer 320 is advanced by a rotational angle 1504, denoted as θ, at each unit time in a clockwise direction 1502 (e.g., driven by the drive cable 210 coupled to the movement device 104). As shown, the transducer 320 provides a scanline 1510a at time t(1), a scanline 1510b at time t(2), and a scanline 1510c at time t(3) with an advancement of an angle 1504 at each time increment. The angular advancement slows down after time t(3). As shown, the transducer 320 provides a scanline 1510d at time t(4) with an angular advancement less than the configured or expected angle 1504. Similarly, the transducer 320 provides a scanline 1510e at time t(5) with an angular advancement less than the angle 1504. At time t(6), the rotational speed speeds up and the transducer 320 provides a scanline 1210f at the expected angular position.

The scheme 1500 performs interpolation, for example, on the scanlines 1510c, 1510d, 1510e, and 1510f to produce the scanlines 1520d and 1520e and remaps the interpolated scanlines 1520d and 1520e according to corresponding expected angular positions. For example, the scanline 1520d is remapped to the angular position as expected for the actual acquired scanline 1510d and the scanline 1520e is mapped to the angular position as expected for the actual acquired scanline 1510e. In the final image construction, the interpolated scanlines 1520d and 1520e at the expected angular positions may be used instead of the received scanlines 1510d and 1510e at the unexpected angular positions.

Typically, NURD is present in a sector of an IVUS image, with most portion of the NURD-affected sector (e.g., the portions 1102 in the image 1110 of FIG. 11) appearing as smeared because of a rotational speed drop, and with shoulders (e.g., the portions 1104 and 1106 in the image 1110 of FIG. 11) of the smeared sector appearing as sharpened because of a rotational acceleration and a rotational deceleration that accompany the speed drop. So, the lateral resampling typically includes lateral downsampling of the smeared part and lateral upsampling of the sharpened shoulders. For the remaining portion of IVUS image outside of the NURD sector, the lateral resampling typically has little or no impact. Thus, while the scheme 1500 illustrates the interpolation and/or lateral resampling over the sector affected by NURD, the interpolation and/or lateral resampling can be generally applied to all scanlines within an image frame. Further, while the scheme 1500 illustrates the transducer 320 rotating in the clockwise direction 1502, similar interpolation and/or resampling mechanisms may be applied to correct and/or reduce NURD when the transducer 320 is configured to rotate in a counter-clockwise direction.

Returning to FIG. 6, the log compression component 660 is coupled to the NURD reduction component 650 and configured to reduce the dynamic range of the signal samples in the NURD reduced signal data 652 for efficient display. For example, the dynamic range of the sample samples may be mapped to a logarithmic curve. In some examples, the log compression component 660 may perform the mapping based on a table lookup, where the table may be encoded with a log compression curve. It should be noted that the mapping table used for log compression at the log compression component 660 may be different from the mapping table used for speckle normalization at the speckle normalization component 710.

The scan conversion component 670 is coupled to the log compression component 660 and configured to perform scan conversion on the signal data 662 output by the log compression component 660 to a suitable display format. In an example, the signal data 662 may be in a polar coordinate and the scan conversion component 670 may convert the signal data 662 into image data 672 in a Cartesian coordinate for display, for example, on the display 108.

While FIG. 6 illustrates the NURD reduction component 650 operating on BB signal data 642, similar NURD reduction mechanisms can be performed on the RF signal data 612 and/or 622.

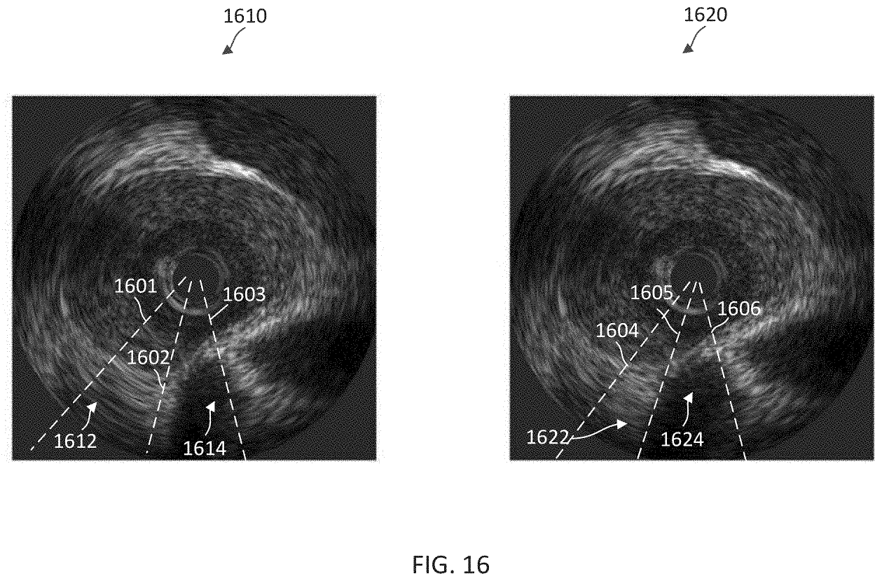

FIG. 16 illustrates NURD reduction in an ultrasound image, according to aspects of the present disclosure. FIG. 16 shows an ultrasound images 1610 and 1620. The images 1610 and 1620 are acquired using a rotational ultrasound device (e.g., the rotational imaging component 110 and/or the transducer 320) in an imaging system (e.g., the systems 100, 200, and/or 600). The image 1610 shows a cross-section of an anatomy (e.g., the anatomy 302) with a smearing sector 1612 (between the dashed lines 1601 and 1602) caused by a slowdown in the rotational speed of the rotational ultrasound device while data is acquired for the image 1610. The image 1620 shows a cross-section of the anatomy constructed using the same acquired data as the image 1610, but NURD reduction is applied as described above, for example, using the NURD reduction component 650.

Comparing the images 1610 and 1620, the smearing sector 1622 (between the dashed lines 1604 and 1605) in the NURD-reduced image 1620 after the NURD reduction is narrower than the smearing sector 1612 in the image 1610 before the NURD reduction. Additionally, the sector 1624 (between the dashed lines 1605 and 1606 adjacent to the smearing sector 1622) in the image 1620 after the NURD reduction is wider than the sector 1614 (between the dashed lines 1602 and 1603 adjacent to the smearing sector 1612) in the image 1610 before the NURD reduction. The narrowing of the smearing sector 1622 is the result of the interpolation and resampling of scanlines performed for the NURD correction. Accordingly, the NURD reduction reduces the effect of NURD. Additionally, the interpolation and resampling cause the widening of the sector 1624 immediately before the smearing sector 1622.

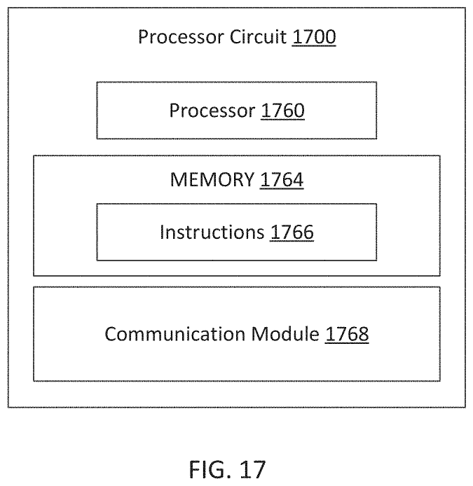

FIG. 17 is a schematic diagram of a processor circuit 1700, according to embodiments of the present disclosure. The processor circuit 1700 may be implemented in the PIM 140 and/or the processing system 106 of FIG. 1. As shown, the processor circuit 1700 may include a processor 1760, a memory 1764, and a communication module 1768. These elements may be in direct or indirect communication with each other, for example via one or more buses.

The processor 1760 may include a CPU, a DSP, an application-specific integrated circuit (ASIC), a controller, an FPGA, another hardware device, a firmware device, or any combination thereof configured to perform the operations described herein, for example, aspects of FIGS. 6-16. The processor 1760 may also be implemented as a combination of computing devices, e.g., a combination of a DSP and a microprocessor, a plurality of microprocessors, one or more microprocessors in conjunction with a DSP core, or any other such configuration.

The memory 1764 may include a cache memory (e.g., a cache memory of the processor 1760), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In an embodiment, the memory 1764 includes a non-transitory computer-readable medium. The memory 1764 may store instructions 1766. The instructions 1766 may include instructions that, when executed by the processor 1760, cause the processor 1760 to perform the operations described herein, for example, aspects of FIGS. 1 and 6-16 and with reference to the PIM 140 and/or the processing system 106 (FIG. 1). Instructions 1766 may also be referred to as code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements.

The communication module 1768 can include any electronic circuitry and/or logic circuitry to facilitate direct or indirect communication of data between the processor circuit 1700, the intraluminal imaging device 102, and/or the display 108. In that regard, the communication module 1768 can be an input/output (I/O) device. In some instances, the communication module 1768 facilitates direct or indirect communication between various elements of the processor circuit 1700 and/or the PIM 140 (FIG. 1) and/or the processing system 106 (FIG. 1).

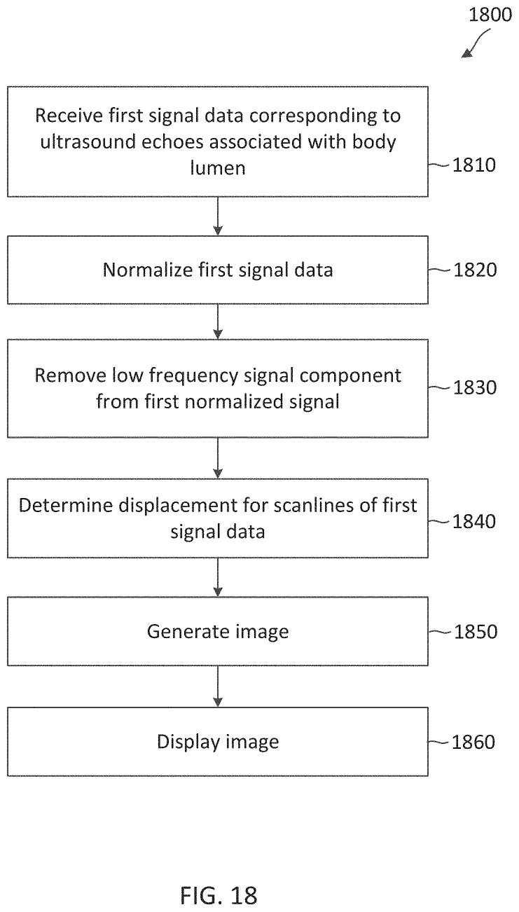

FIG. 18 is a flow diagram of an ultrasound imaging method 1800 with NURD reduction, according to aspects of the disclosure. Steps of the method 1800 can be executed by the systems 100, 200, and/or 600, for example, by a processor circuit such as the processor circuit 1700, and/or other suitable component such as the imaging component 110, the PIM 140, and/or the processing system 106, and/or any suitable combination of components in the system 100, 200 and/or the system 600. The method 1800 may employ similar mechanisms as in the systems 100, 200, and 600 described with respect to FIGS. 1, 2, and 6, respectively, the NURD reduction component 650 with respect to FIG. 7, the schemes 800, 900, 1200, 1300, and 1500 described with respect to FIGS. 8, 9, 12, 13, and 15, respectively. As illustrated, the method 1800 includes a number of enumerated steps, but embodiments of the method 1800 may include additional steps before, after, and in between the enumerated steps. In some embodiments, one or more of the enumerated steps may be omitted or performed in a different order.

At step 1810, the method 1800 includes receiving first signal data (e.g., the signal data corresponding to ultrasound echoes associated with a body lumen. The first signal data may be received at a processor circuit (e.g., the processor circuit 1700) in communication with an intraluminal imaging device (e.g., the rotational intraluminal imaging device 102) including an ultrasound transducer (e.g., the transducer 320) positioned at distal portion of a rotational, flexible elongate drive cable (e.g., the driveshaft 210). The processor circuit may be located at a PIM similar to the PIM 140 or a processing system similar to the processing system 106. The first signal data may be similar to the signal data 602 and/or 642. The body lumen may be a vessel similar to the anatomy 302.

At step 1820, the method 1800 includes normalizing the first signal data by adjusting an intensity range of the first signal data, for example, by utilizing the speckle normalization component 710.

At step 1830, the method 1800 includes determining second signal data (e.g., the signal data 722) based on a removal of a low frequency signal component from the first normalized signal data (e.g., the signal data 712), for example, by utilizing the background subtraction component 720.

At step 1840, the method 1800 includes determining a displacement (e.g., the angular spacing 752) for one or more scanlines (e.g., the scanlines 410, 810, and/or 1210) of the first signal data based on the second signal data, for example, by utilizing the lateral displacement estimation component 750.

At step 1850, the method 1800 includes generating an image (e.g., the images 1620) of the body lumen based on the displacement.

At step 1860, the method 1800 includes outputting the image to a display (e.g., the display 108) in communication with the processor circuit.

In an embodiment, the displacement is associated with a rotational motion of the ultrasound transducer.

In an embodiment, the normalizing the first signal data includes applying a reference intensity map to the first signal data. The determining the second signal data includes applying at least one of a high-pass filter operation (e.g., as shown in the scheme 800) or an averaging operation and subtraction (e.g., as shown in the scheme 900) to the first normalized signal data.

In an embodiment, the method 1800 further includes performing speckle evaluation, for example, by utilizing the speckle evaluation component 730. For example, a first scanline of the one or more scanlines includes a first sample (e.g., the sample 1212a, 1212b, or 1212c) at a first imaging depth and a second sample at a second imaging depth. The evaluation includes determining a first quantity (e.g., an integer count) of consecutive samples at the first imaging depth across neighboring scanlines of the first scanline (e.g., within the window 1210a) based on an intensity threshold (e.g., a background intensity value). The evaluation further includes determining a second quantity of consecutive samples at the second imaging depth across neighboring scanlines of the first scanline (e.g., within the window 1210b) based on the intensity threshold. The evaluation further includes determining a metric (e.g., the metrics 732, 1220, and/or 1320) for the first scanline based on the first quantity and the second quantity. In an embodiment, the metric is determined by applying at least one of an accumulation operation (e.g., as shown in the scheme 1200), an averaging operation (e.g., as shown in the scheme 1300), or a median operation (e.g., as shown in the scheme 1300), to the first quantity and the second quantity.

In an embodiment, after determining a metric (e.g., the metrics 732, 1220, and/or 1320) for each scanline of the one or more scanlines based on the second signal data, the method 1800 includes determining a rotational parameter (e.g., the rotational speed 742) of the ultrasound transducer associated with the one or more scanlines based on corresponding metrics, for example, by utilizing the rotational speed mapping component 740. The determining the displacement includes determining a lateral displacement (e.g., the angular spacing 752) for the one or more scanlines based on the rotational parameter, for example, by utilizing the lateral displacement estimation component 750. The image can be generated by resampling the one or more scanlines based on the lateral displacement, for example, by utilizing the interpolation and lateral resampling component 760.

In an embodiment, the steps 1810 and 1850 correspond to the main imaging path, where the ultrasound echo signal data 642 output by the LPF 640 is being processed by the interpolation/lateral resampling component 760 as shown in FIG. 7. The steps 1820-1840 correspond to the NURD evaluation path where the ultrasound echo signal data 642 is being processed by one or more of the speckle normalization component 710, the background subtraction component 720, the speckle evaluation component 730, the rotational speed mapping component 740, or the lateral displacement estimation component 750. In some embodiments, all or a portion of the imaging path and the NURD evaluation path can be different.

Aspects of the present disclosure can provide several benefits. For example, the enhancement of the speckle pattern via the intensity normalization (e.g., perform by the speckle normalization component) and the removal of the low frequency intensity variation signal components (e.g., perform by the background subtraction component) can remove or reduce the dependencies of signal and/or noise characteristics in the underlying image from the NURD reduction processing. Accordingly, the NURD detection and/or correction can provide a more accurate NURD estimate in an ultrasound image frame and thus may provide a higher quality ultrasound image with improved NURD reduction. The direct counting with the accumulation-based metrics and/or the histogram-based metrics used for the speckle evaluation can provide a lower computational loading compared to cross correlation-based and/or frequency spectra-based NURD reduction algorithms and thus may be suitable for implementation on lower cost and/or lower performance processing devices.

Persons skilled in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. An ultrasound imaging system, comprising:
an intraluminal imaging device configured to be positioned within a body lumen of a patient, the intraluminal imaging device comprising:
a rotatable, flexible elongate drive cable; and
an ultrasound transducer disposed at a distal portion of the drive cable, the ultrasound transducer configured to transmit ultrasound energy into the body lumen and to receive ultrasound echoes associated with the body lumen; and
a processor circuit in communication with the intraluminal imaging device and configured to:
receive, from the ultrasound transducer, first signal data corresponding to the received ultrasound echoes;
normalize the first signal data by adjusting an intensity range of the first signal data;
determine second signal data based on a removal of a low frequency signal component from the first normalized signal data;
determine a displacement for one or more scanlines of the first signal data based on the second signal data;
generate an image of the body lumen based on the displacement; and
output the image to a display in communication with the processor circuit.

2. The ultrasound imaging system of claim 1, wherein the displacement is associated with a rotational motion of the ultrasound transducer.

3. The ultrasound imaging system of claim 1, wherein the processor circuit configured to normalize the first signal data is further configured to:
apply a reference intensity map to the first signal data.

4. The ultrasound imaging system of claim 3, wherein the processor circuit configured to generate the image is further configured to:
perform brightness-mode (B-mode) processing on the first signal data based on a log compression map different from the reference intensity map.

5. The ultrasound imaging system of claim 1, wherein the processor circuit configured to determine the second signal data is further configured to:
apply a high-pass filter to the first normalized signal data.

6. The ultrasound imaging system of claim 1, wherein the processor circuit configured to determine the second signal data is further configured to:
determine an average intensity for at least a portion of the first normalized signal data; and
subtract the average intensity from the first normalized signal data.

7. The ultrasound imaging system of claim 1, wherein the processor circuit is further configured to:
determine a metric for each scanline of the one or more scanlines based on the second signal data.

8. The ultrasound imaging system of claim 7, wherein a first scanline of the one or more scanlines includes a first sample at a first imaging depth and a second sample at a second imaging depth, and wherein the processor circuit configured to determine the metric is further configured to:
determine a first quantity of consecutive samples at the first imaging depth across neighboring scanlines of the first scanline comprising a first intensity value;
determine a second quantity of consecutive samples at the second imaging depth across neighboring scanlines of the first scanline comprising the first intensity value; and
determine the metric for the first scanline based on the first quantity and the second quantity.

9. The ultrasound imaging system of claim 8, wherein the processor circuit configured to determine the metric is further configured to:
apply at least one of an accumulation operation, an averaging operation, or a median operation to the first quantity and the second quantity.

10. The ultrasound imaging system of claim 8, wherein the first intensity value is associated with a background intensity threshold.

11. The ultrasound imaging system of claim 7, wherein the processor circuit is further configured to:
determine a rotational parameter of the ultrasound transducer associated with the one or more scanlines based on a corresponding metric.

12. The ultrasound imaging system of claim 11, wherein the processor circuit configured to determine the displacement is further configured to:
determine a lateral displacement for the one or more scanlines based on the rotational parameter.

13. The ultrasound imaging system of claim 1, wherein the processor circuit configured to generate the image is further configured to:
resample the one or more scanlines based on the displacement.

14. The ultrasound imaging system of claim 13, wherein the processor circuit configured to generate the image is further configured to:
perform brightness-mode (B-mode) processing on the one or more resampled scanlines.

15. A method of ultrasound imaging, comprising:
receiving, at a processor circuit in communication with an intraluminal imaging device including an ultrasound transducer positioned at distal portion of a rotational, flexible elongate drive cable, first signal data corresponding to ultrasound echoes associated with a body lumen;
normalizing the first signal data by adjusting an intensity range of the first signal data;
determining second signal data based on a removal of a low frequency signal component from the first normalized signal data;
determining a displacement for one or more scanlines of the first signal data based on the second signal data;
generating an image of the body lumen based on the displacement; and
outputting the image to a display in communication with the processor circuit.

16. The method of claim 15, wherein the displacement is associated with a rotational motion of the ultrasound transducer.

17. The method of claim 15, wherein:
the normalizing the first signal data includes:
applying a reference intensity map to the first signal data, and the determining the second signal data includes:
applying at least one of a high-pass filter operation or an averaging operation to the first normalized signal data.

18. The method of claim 15, wherein a first scanline of the one or more scanlines includes a first sample at a first imaging depth and a second sample at a second imaging depth, and wherein the method further comprises:
determining a first quantity of consecutive samples at the first imaging depth across neighboring scanlines of the first scanline based on an intensity threshold;
determining a second quantity of consecutive samples at the second imaging depth across neighboring scanlines of the first scanline based on the intensity threshold; and
determining a metric for the first scanline based on the first quantity and the second quantity.

19. The method of claim 18, wherein the determining the metric includes:
applying at least one of an accumulation operation, an averaging operation, or a median operation to the first quantity and the second quantity.

20. The method of claim 15, further comprising:
determining a metric for each scanline of the one or more scanlines based on the second signal data;
determining a rotational parameter of the ultrasound transducer associated with the one or more scanlines based on corresponding metrics,
wherein the determining the displacement includes:
determining a lateral displacement for the one or more scanlines based on the rotational parameter; and
wherein the generating the image includes:
resampling the one or more scanlines based on the lateral displacement.
